(19) United States
(12) Patent Application Publication
(10) Pub. No.: US 2008/0215149 A1
(43) Pub. Date: Sep. 4, 2008

(54) Lightweight Silicone Implant and Manufacturing Method Thereof

(76) Inventors: Eun Hyun Bae, Seoul (KR); Byung Ho Lee, Koyang-si (KR)

Correspondence Address: PARK LAW FIRM, 3255 WILSHIRE BLVD, SUITE 1110, LOS ANGELES, CA, 90010, US

(21) Appl. No.: 11/848,120

(22) Filed: Aug. 30, 2007

(30) Foreign Application Priority Data
Oct. 20, 2006 (KR) 10-2006-0102434

(51) Int. Cl. A61F 2/02

(52) U.S. Cl. 623/11.11

(57) ABSTRACT

Disclosed herein are a lightweight silicone implant which is manufactured by filling a foamed silicone sponge and/or vacant silicone capsules in a silicone bag or a cohesive gel bag and sealing the bag, and a method for manufacturing the same.

### TECHNICAL FIELD

The present invention relates to a lightweight silicone implant and a manufacturing method thereof, and more particularly to a lightweight silicone implant which is manufactured by filling a foamed silicone sponge and/or vacant silicone capsules in a silicone bag or a cohesive gel bag and sealing the bag, and a method for manufacturing the lightweight silicone implant.

### BACKGROUND ART

As a modern society is diversified at a rapid pace, interest and standards regarding an outward appearance are changing. Accordingly, in order to enable a person to recover confidence in a social life and lead a better life, plastic surgery for changing and complementing the outward appearance now prevails.

Silicone is commonly used in plastic surgery. This is because silicone has features of being cheap, harmless to a human body, capable of being carved precisely so as to be accurately matched with shape of a bone, and not being deformed in size or shape after the operation. A silicone implant used in the plastic surgery is classified into four types as follows.

A first type is a saline bag which is constituted by a silicone bag filled with saline. The implant using the saline has an advantage of being cheap, but has a shortcoming that the saline leaks due to bursting or splitting of the bag during use.

A second type is a silicone gel bag which is constituted by a silicone bag filled with fluid silicone gel. The silicone gel feels relatively soft. However, when the silicone gel leaks due to bursting of the bag, the gel particles are dispersed and absorbed in a human body, and is harmful to the human body. In reality, it has been reported that the silicone gel bag manufactured by Dow Corning Corporation, USA, burst and the leaked gel particles had an injurious effect on the human body.

A third type is a cohesive gel bag which is constituted by a silicone bag filled with cohesive gel. The cohesive gel is to complement the shortcoming of the silicone gel. The cohesive gel is known as being the best product in terms of soft feeling and safety. However, the cohesive gel has not received the formal approval from the US FDA (Food and Drug Administration), but has only conditional approval. Some countries except for Korea still do not approve the cohesive gel. Also, the cohesive gel is very expensive. Further, it is likely difficult to get the approval because of the aforesaid accident of the silicone gel bag manufactured by Dow Corning Corporation, USA.

A fourth type is a silicone block which is manufactured by carving the common silicone lump as needed. The silicone block is very easy to be carved by a surgeon. However, because the silicone block is hard and heavy, it feels heterogeneous from the recipient's body and does not provide convenience in use.

Besides the above four types of implants, there is a double lumen bag which is devised by combining the advantages of the saline and the silicone gel. The double lumen bag is constituted by the saline bag filled with the silicone gel or the silicone gel bag filled with the saline. The above-described types of implants are adequately used for plastic surgical operation according to operation purposes with respect to diverse body regions such as a nose, a forehead, a jaw, a cheek, a breast, a hip, a calf and the like. However, as volume of the above-described types of implants is increased, weight becomes also very heavy.

Accordingly, in case of the narrow body region such as the nose, the cheek or the jaw in which the relatively small and light implant is inserted, there is no particular inconvenience in daily life. But, in case of the broad body region such as the hip, the breast, the forehead or the calf in which the relatively large and heavy implant is inserted, the recipient feels it is heterogeneous from her/his body or has inconvenience in actions.

For example, in case of the nose correction, because the small implant is inserted between the nasal bone and the periosteum, the implant can be securely fixed and does not deviate from its original position after the plastic surgery. However, in case of the breast or hip correction, because the relatively large implant cannot be inserted between the bone and the periosteum, the implant must be inserted in muscles. Such a large and heavy implant may deviate from its original position after the plastic surgery. Especially, after the plastic surgery of inserting the implant in the hip, when the recipient sits on a chair or lies on her/his back, the bag of the implant may be burst by the recipient's weight, and thus material filled in the bag may leak and/or the implant may deviate from its initial position fixed in the plastic surgery.

Also, because the conventional implant for the hip correction does not have sufficient elasticity, when an external force is applied to the implant in any direction according to the recipient [symbol] posture or actions, the force-receiving portion of the implant is contracted and the opposite portion of the implant is expanded. Therefore, the pressure exerted on the implant-inserted region is increased, and the recipient feels uncomfortable.

### DISCLOSURE

### Technical Problem

Therefore, the present invention has been made in view of the above problems, and it is an object of the present invention to provide a lightweight silicone implant which does not move by an external force, has good adaptability to a human body, feels soft like flesh, and has high elasticity, so as to be effectively inserted in a relatively large body region.

It is another object of the present invention to provide a method for manufacturing the above lightweight silicone implant.

### Technical Solution

In accordance with an aspect of the present invention, the above and other objects can be accomplished by the provision of a lightweight silicone implant characterized in that: the lightweight silicone implant is manufactured by filling a foamed silicone sponge in a silicone bag or a cohesive gel bag and sealing the bag.

In accordance with another aspect of the present invention, there is provided a lightweight silicone implant characterized in that: the lightweight silicone implant is manufactured by filling vacant silicone capsules in a silicone bag or a cohesive gel bag and sealing the bag.

### ADVANTAGEOUS EFFECTS

According to a lightweight silicone implant and a manufacturing method thereof in accordance with the present invention, since the lightweight silicone implant of the present invention feels soft and elastic just like flesh, beyond the limitation of the conventional medical silicone, it can provide a recipient with intense satisfaction.

Also, the lightweight silicone implant can be securely fixed and does not deviate from its original position after the plastic surgery.

Further, although an external force is exerted on a portion of the silicone implant, only this specific portion is just contracted, but other portions of the silicone implant are not deformed or expanded.

### DESCRIPTION OF DRAWINGS

The above and other objects, features and other advantages of the present invention will be more clearly understood from the following detailed description taken in conjunction with the accompanying drawings, in which:

FIGS. 1 to 3 are plan views and side views showing various conventional implants;

FIG. 4 is a schematic view showing a human hip in which an implant is inserted;

FIG. 5 is a photograph showing appearances before and after inserting an implant in the human hip;

FIG. 6 is a photograph showing a conventional breast implant,

FIG. 7 is a photograph showing a conventional hip implant;

FIG. 8 is a photograph showing a silicone sponge and its elasticity according to the present invention;

FIG. 9 is a photograph showing a cohesive gel and a cohesive gel bag;

FIG. 10 is a cross-sectional view showing an implant in accordance with a preferred embodiment of the present invention which uses a foamed silicone sponge and a silicone bag;

FIG. 11 is a cross-sectional view showing an implant in accordance with another preferred embodiment of the present invention which uses a foamed silicone sponge and a cohesive gel bag;

FIG. 12 is a cross-sectional view showing an implant in accordance with yet another preferred embodiment of the present invention which uses a vacant silicone capsule and a silicone bag;

FIG. 13 is a cross-sectional view showing an implant in accordance with yet another preferred embodiment of the present invention which uses a vacant silicone capsule and a cohesive gel bag;

FIG. 14 is a schematic view showing a manufacturing process of a silicone sponge according to the present invention which includes a primary curing step of compressing and foaming sponge rubber material and a secondary curing step of drying a foamed sponge; and

FIG. 15 is a schematic view showing a manufacturing process of a lightweight silicone implant according to the present invention.

### BEST MODE

Now, preferred embodiments of the present invention will be described in detail with reference to the annexed drawings.

The present invention provides a lightweight silicone implant which is manufactured by filling a foamed silicone sponge in a silicone bag or a cohesive gel bag and sealing the bag.

Preferably, a method for manufacturing the above lightweight silicone implant includes the steps of: (a) forming a foamed silicone sponge having a desired shape by using a silicone foam resin; (b) coating a spray liquid adhesive on a surface of the silicone sponge; and (c) adhering a silicone bag or a cohesive gel bag having a predetermined thickness to the surface of the silicone sponge on which the adhesive is coated and adequately shaping the bag. It is preferable to use a pre-form having the desired shape in the foaming of the silicone resin, and set the thickness of the silicone bag or the cohesive gel to be in the range of 0.3 mm to 5 mm.

Also, the present invention further provides a lightweight silicone implant which is manufactured by putting a vacant silicone capsule into a silicone bag or a cohesive gel bag and sealing the bag.

Preferably, a method for manufacturing the above lightweight silicone implant includes the steps of: (a) making vacant silicone capsules having a desired shape by combining a plurality of small silicone capsules and small silicone pieces at high temperature; (b) coating a spray liquid adhesive on a surface of the vacant silicone capsules; and (c) adhering a silicone bag or a cohesive gel bag having a predetermined thickness to the surface of the vacant silicone capsules on which the adhesive is coated and adequately shaping the bag.

The present invention provides a lightweight silicone implant which has good adaptability to a human body, feels soft like flesh, and has high elasticity, for the purpose of reconstructive plastic surgery or cosmetic plastic surgery.

In the lightweight silicone implant according to the present invention, the cohesive gel bag is made by filling the cohesive gel into the silicone bag, and the silicone form (the bag-shaped form) or the cohesive gel bag has a thickness of 0.3 mm to 5.0 mm. At this time, the thickness of the silicone implant or the cohesive gel bag is varied as needed or according to the body region. For example, the thickness of a portion of the implant which faces an outer skin is about one-tenth to three-tenths of the thickness of the other portion of the implant. By doing so, the implant can feel softer at the outer skin.

Also, in the lightweight silicone implant according to the present invention, the silicone form or the cohesive gel bag is coupled to the foamed silicone sponge and/or the silicone capsules by use of the spray liquid adhesive.

As described above, the conventional silicone implant had shortcomings that the weight becomes very heavy as the volume is increased and does not feel soft (feels heterogeneous), thereby causing inconvenience in daily actions and gives an unpleasant feeling. To solve this problem of the conventional silicone implant, the inventors of the present invention suggest that the lightweight silicone sponge and/or the silicone capsules substitute the conventional silicone implant. However, the silicone sponge and/or the silicone capsules are disadvantageous of being unsafe and requiring a cover because they are too weak and the surface is rough. Therefore, a method of adhering the harder silicone form or the cohesive gel bag to the surface of the silicone sponge and/or the silicone capsules has been developed.

Hereinafter, the lightweight silicone implant and the manufacturing method thereof according to the present invention will be described in detail by dividing into a method using the foamed silicone resin (sponge) and a method using the silicone capsules.

### Method of Using the Silicone Resin

### 1st Step: Foaming the Silicone Resin (Sponge)

The silicone resin is formed by a common casting process using a mold. The porous silicone foam resin is filled in the mold having a desired shape by injection, and compressed at high temperature. And, the silicone resin is foamed at high temperature. At this time, in order to increase foaming efficiency of the silicone resin, it is preferable to use the pre-form.

### 2nd Step: Coating the Spray Liquid Adhesive

Although the foamed silicone resin has a low specific gravity, low weight and does no harm to the human body, because the foamed silicone resin absorbs water due to its porosity, the foamed silicone resin is enveloped by the silicone bag or the cohesive gel bag which is proven to be safe and harmless to the human body. In order to securely adhere the silicone bag to the silicone resin (sponge), the spray liquid adhesive which is harmless to the human body is coated on the surface of the silicone resin.

### 3rd Step: Adhering the Silicone Bag or the Cohesive Gel Bag

The silicone bag or the cohesive gel bag having a proper thickness is adhered to the surface of the silicone resin, and compressed at high temperature. As a result, the lightweight silicone implant satisfying the purpose of the present invention can be manufactured. At this time, the cohesive gel bag is a bag-shaped cohesive gel on which a separate silicone material is coated. Also, the silicone bag or the cohesive gel bag may be manufactured by separately providing an oval upper part and an oval lower part and coupling them.

### Method of Using the Vacant Silicone Capsules

### 1st Step: Manufacturing the Vacant Silicone Capsules

The silicone capsules are formed by a common casting process using a mold. A plurality of small silicone capsules and small silicone pieces are filled in the mold having a desired shape by injection, and compressed at high temperature. The capsules are filled with air, and are not burst by a human body weight or an unintended external force.

### 2nd Step: Coating the Spray Liquid Adhesive

Since it is the same as the step of the method using the foamed silicone resin, the description thereof is omitted.

### 3rd Step: Adhering the Silicone Bag or the Cohesive Gel Bag

Since it is the same as the step of the method using the foamed silicone resin, the description thereof is omitted.

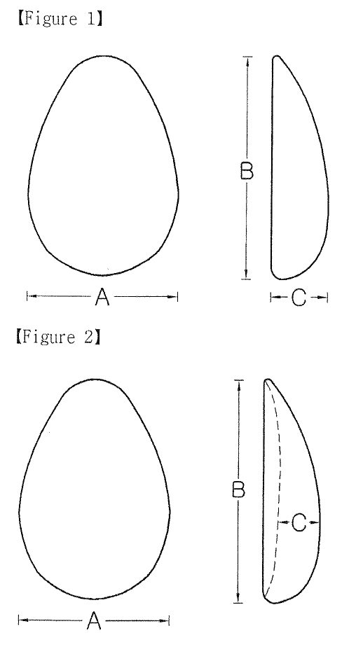

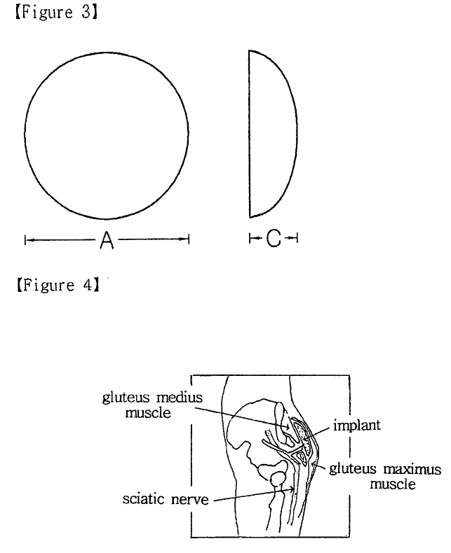

The present invention will be more clearly understood from FIGS. 1 to 9.

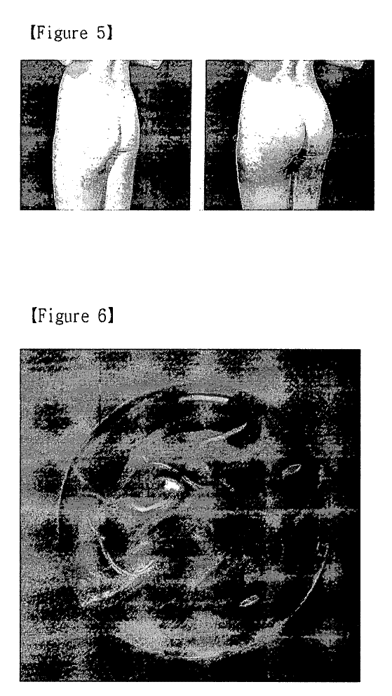

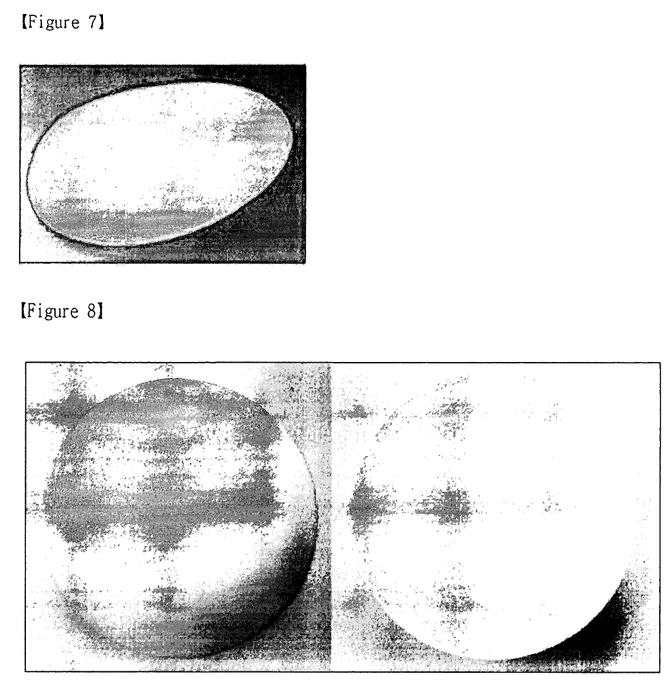

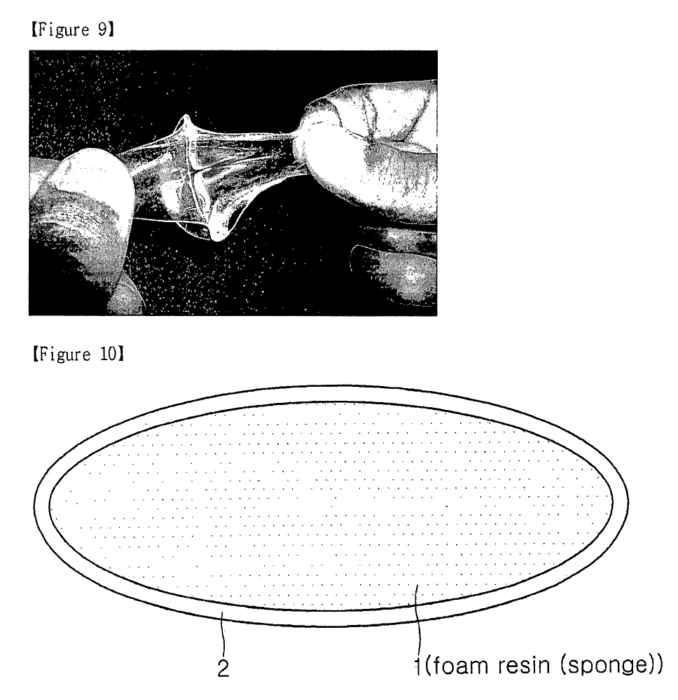

FIGS. 1 to 3 are plan views and side views showing various conventional implants. As shown in the drawings, the implant typically has an oval or circular shape. In the drawings, a reference character A means a minor axis, B means a major axis, and C means a height. FIG. 4 is a schematic view showing a human hip in which the implant is inserted. As shown in the drawing, the silicone implant is inserted in a so-called “submuscular pocket”, i.e., a sub-gluteal muscle space surrounded by a gluteus medius muscle, a gluteus maximus muscle and a sciatic nerve. FIG. 5 is a photograph showing appearances before and after inserting the implant in the hips of a 24-year-old recipient. More particularly, the left photograph shows the appearance before inserting the implant in the hips, and the right photograph shows the appearance when six months have passed since the circular silicone implant is inserted in the sub-gluteal muscle space. FIG. 6 is a photograph showing a conventional breast implant, FIG. 7 is a photograph showing a conventional hip implant, FIG. 8 is a photograph showing the silicone sponge and its elasticity according to the present invention, and FIG. 9 is a photograph showing the cohesive gel and the cohesive gel bag (the bag-shaped cohesive gel on which a separate silicone material is coated) according to the present invention.

Since the silicone sponge of the present invention depicted in FIG. 8 is porous and has a considerably low specific gravity, it has an extremely high elasticity and feels soft like flesh. Especially, although an external force is exerted on a portion of the silicone sponge of the present invention, the change of volume at other portions thereof is not generated at all.

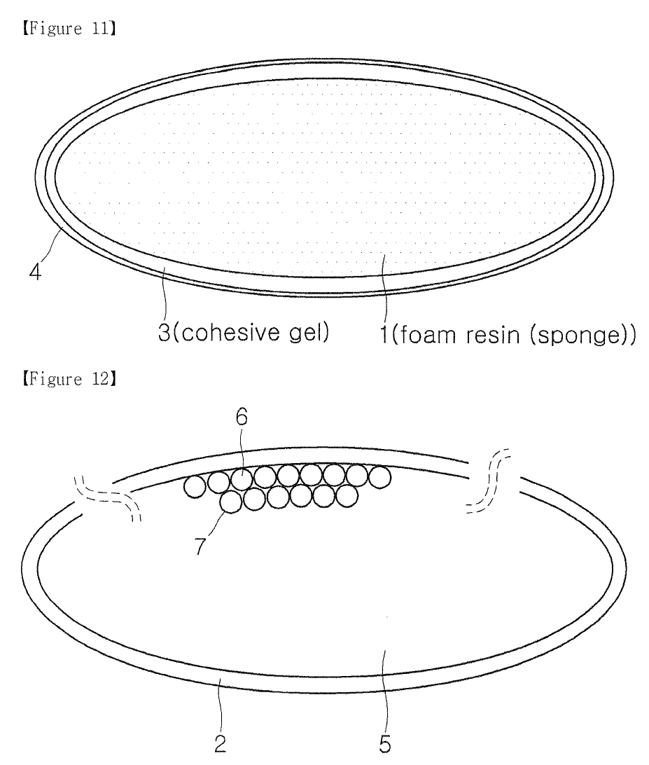

FIG. 10 is a cross-sectional view showing the implant according to a preferred embodiment of the present invention. As shown in the drawing, the implant is constituted by the silicone bag 2 (manufactured by Nagor Ltd., England) made from a common medical silicone material, and the foamed silicone sponge 1 filled in the silicone bag. Since such a lightweight silicone implant is harmless to the human body and can be formed in an adequate shape as needed, it can be naturally matched with the body shape of the recipient. Also, the lightweight silicone implant can minimize the occurrence of side effects like inflammation due to reaction to a foreign substance and maintain the stability in shape because it is not deformed or its height is not reduced during a long period after the plastic surgery. Further, since the lightweight silicone implant of the present invention feels soft and elastic just like flesh, beyond the limitation of the conventional medical silicone, it can provide the recipient with intense satisfaction.

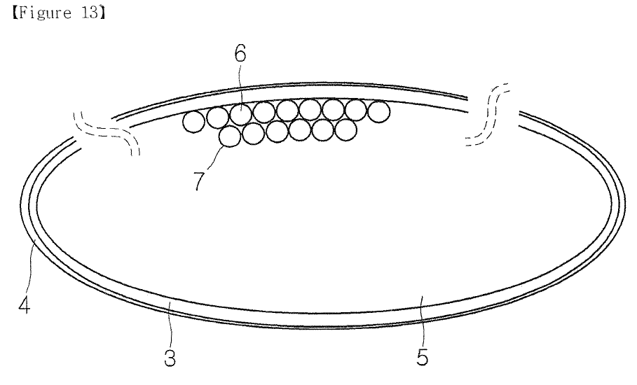

FIG. 11 is a cross-sectional view showing the implant according to another preferred embodiment of the present invention. As shown in the drawing, the implant is constituted by the cohesive gel bag (“CoGel” manufactured by Nagor Ltd., England), and the foamed silicone sponge 1 filled in the cohesive gel bag. The cohesive gel bag is formed by containing the cohesive gel 3 in the silicone bag 4. FIG. 12 is a cross-sectional view showing the implant according to yet another preferred embodiment of the present invention. As shown in the drawing, the implant is constituted by the medical silicone bag 2 and the vacant silicone capsules 6 filled in the silicone bag 2. At this time, the silicone bag 2 is formed in the desired shape in advance, and then the plurality of the vacant silicone capsules 6 are filled in the silicone bag 2. FIG. 13 is a cross-sectional view showing the implant according to yet another preferred embodiment of the present invention. As shown in the drawing, the implant is constituted by the cohesive gel bag 4 and the vacant silicone capsules 6 filled in the cohesive gel bag 4. Since the embodiments shown in FIGS. 11 to 13 have the same or similar functions as/to the embodiment shown in FIG. 10, the description thereof will be omitted.

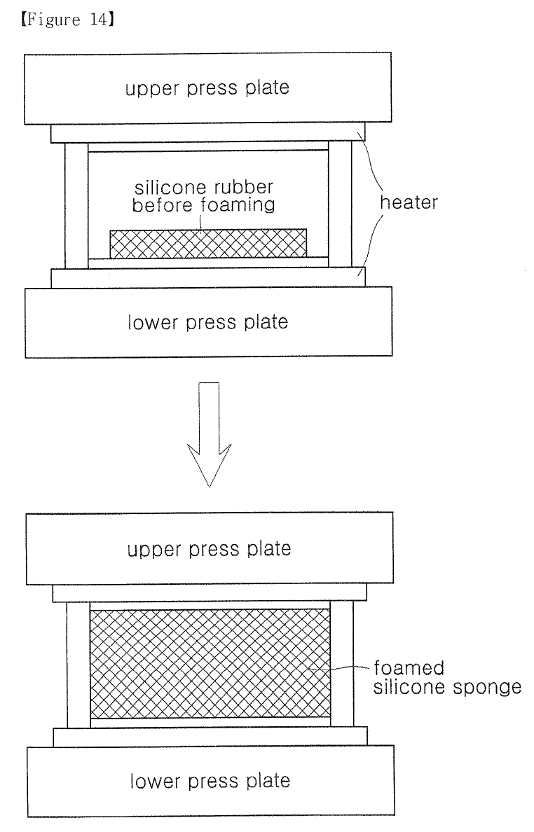

Hereinafter, an embodiment of the method for manufacturing the lightweight silicone implant according to the present invention will be described with reference to FIG. 14.

First, a mold having a desired shape is produced to manufacture a silicone sponge. A compound silicone rubber material before being foamed is injected into the mold, and compressed and foamed by using an upper press plate disposed on the mold and a lower press plate disposed under the mold (a primary curing process). In order to perform the compressing and foaming operations at high temperature, it is preferable to mount heaters under the upper press plate and on the lower press plate. After the silicone rubber is sufficiently foamed to form the silicone sponge, the foamed silicone sponge is dried in a hot air drying furnace, for example (a secondary curing process).

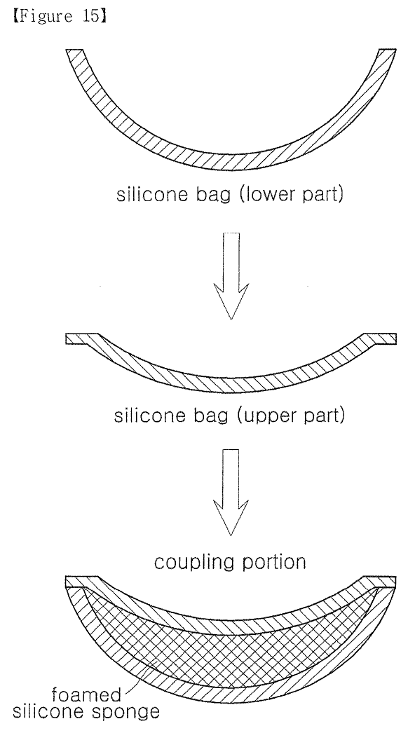

As shown in FIG. 15, the silicone sponge manufactured as above is put in the silicone bag which is formed corresponding to the shape of the silicone sponge. Preferably, the silicone bag includes an upper part and a lower part which are coupled to each other. By using a spray liquid adhesive which is harmless to the human body, the edge of the upper part is adhered to the edge of the lower part, so that the silicone bag is sealed to prevent the leakage of the silicone sponge.

The silicone sponge manufactured as above has various features suitable to be used for the lightweight silicone implant (see the following table 1). The silicone sponge used in the silicone implant of the present invention is sharply increased in volume and decreased in specific gravity from being foamed. Nevertheless, since the foamed silicone sponge keeps the properties (hardness and tensile strength) of the rubber, the foamed silicone sponge can be used for the silicone implant. For example, while a mass of the silicone bag of 100 ml is about 175 g, a mass of the silicone sponge of 100 ml is only about 95 g. The lightweight silicone implant of the present invention manufactured as above can be inserted in the hip, the breast, the forehead, the calf and the like.

Hereinafter, the present invention will be described more in detail.

### EMBODIMENT 1

### Manufacture of the Lightweight Silicone Implant

As one embodiment of the present invention, the lightweight silicone implant is manufactured by filling the silicone sponge in the silicone bag.

<1-1> Manufacture of the Silicone Sponge

In order to manufacture the silicone sponge, a mold having a desired shape is first produced. A compound silicone rubber material before being foamed is injected into the mold, and compressed and foamed at temperature of 165° C. by using an upper press plate disposed on the mold and a lower press plate disposed under the mold (a primary curing process). In order to perform the compressing and foaming operations at high temperature of 165° C. heaters are mounted under the upper press plate and on the lower press plate. At this time, the silicone rubber is made in a pre-form type having a desired shape. After the silicone rubber is sufficiently foamed to form the silicone sponge, the foamed silicone sponge is dried in a hot air drying furnace for about 10 minutes (a secondary curing process) (see FIG. 14). Because the surface of the silicone sponge is rough, it is polished as shown in FIG. 8. This is because as the surface of the silicone sponge is smoother, adhering efficiency of the silicone sponge and the silicone bag is increased.

<1-2> Manufacture of the Silicone Implant

The silicone sponge manufactured in the above step <1-1> is put in the silicone bag which is formed corresponding to the shape of the silicone sponge (see FIG. 15). The silicone bag includes an upper part and a lower part which are coupled to each other. At this time, the silicone bag may be manufactured to have the constant thickness. Preferably, in this embodiment, the thickness of the lower part of the silicone bag is about 0.3 mm, and the thickness of the upper part of the silicone bag is about 3 mm. By using a spray liquid adhesive which is harmless to the human body, the edge of the upper part is adhered to the edge of the lower part, so that the silicone bag is sealed to prevent the leakage of the silicone sponge.

### EMBODIMENT 2

### Inspection of Properties of the Silicone Sponge

In order to inspect whether the silicone sponge manufactured in the above step <1-1> of the embodiment 1 is adequate to be used for the lightweight silicone implant, the properties of the silicone sponge are inspected (see Table 1). As seen from the table 1, the silicone sponge of the present invention is increased by four times in volume and sharply decreased in specific gravity from being foamed, and the foamed silicone sponge keeps the properties (hardness and tensile strength) of the rubber. Accordingly, it is confirmed that the above silicone sponge can be effectively used for the silicone implant. In particular, while a mass of the silicone bag of 100 ml is about 175 g, a mass of the silicone sponge of 100 ml is only about 95 g. Like this, since the silicone sponge of the present invention has very low specific gravity, the silicone implant does not move and is fixed in its position even when an external force due to strenuous exercise is exerted thereon. Further, since the silicone sponge of the present invention has good elasticity, although an external force is exerted on a portion of the silicone implant, only this specific portion is contracted, but other portions of the silicone implant are not deformed or expanded.

TABLE 1

| | | | Foaming |
|---|---|---|---|
| Silicone Material | | KE 903^{F} | Agent |
| (1) Before Curing | Appearance | Milky White | White |
| | | Translucent | |
| | Specific Gravity (25° C.) | 1.11 | 0.8 |
| | Plasticity | 180 | |
| (2) Formed Sponge | Foaming Magnitude | 400% | |
| | Hardness after 1st | 30 kg/cm² | |
| | Curing | | |
| | Hardness after 2nd | 40 kg/cm² | |
| | Curing | | |
| (3) Rubber Sample | Hardness | 40 kg/cm² | |
| | Tensile Strength | 75 kg/cm² | |
| | Elongation | 470% | |

| | |
|---|---|
| Compression/ | 22 hours at 180° C. |
| Durability | |

### INDUSTRIAL APPLICABILITY

As apparent from the above description, the lightweight silicone implant in accordance with the present invention is adequate to be inserted in the hip, the breast, the forehead, the calf and the like. For example, when being inserted in the hip, the lightweight silicone implant can be inserted under fascia or between muscles. It is preferable that the lightweight silicone implant is inserted between a gluteus medius muscle and a gluteus maximus muscle rather than be inserted under the fascia, so as to prevent inflammation from being generated or liquid from accumulating in the muscles.

Since the lightweight silicone implant of the present invention does not move by an external force, stably maintains its original shape, and feels soft and elastic just like flesh, it can provide the recipient with intense satisfaction. Further, the lightweight silicone implant has good adaptability to the human body, and can minimize the occurrence of side effects like inflammation due to reaction to a foreign substance, as such it can be effectively used in the plastic surgical operation for the relatively large body region, such as the hip, the breast, the forehead, the calf and the like.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A lightweight silicone implant characterized in that:
the lightweight silicone implant is manufactured by filling a foamed silicone sponge in a silicone bag or a cohesive gel bag and sealing the bag.

2. A lightweight silicone implant characterized in that:
the lightweight silicone implant is manufactured by filling vacant silicone capsules in a silicone bag or a cohesive gel bag and sealing the bag.

3. The lightweight silicone implant according to claim 1, wherein the cohesive gel bag includes a silicone bag and a cohesive gel contained in the silicone bag.

4. The lightweight silicone implant according to claim 1, wherein the silicone bag or the cohesive gel bag has a thickness of 0.3 mm to 5 mm.

5. The lightweight silicone implant according to claim 4, wherein the thickness of the silicone bag or the cohesive gel bag is partially varied according to a body region.

6. The lightweight silicone implant according to claim 1, wherein the silicone bag or the cohesive gel bag is coupled to the foamed silicone sponge or the silicone capsules by use of a spray liquid adhesive.

7. A method for manufacturing a lightweight silicone implant comprising the steps of:
(a) forming a foamed silicone sponge having a desired shape by using a silicone foam resin;
(b) coating a spray liquid adhesive on a surface of the silicone sponge; and
(c) adhering a silicone bag or a cohesive gel bag having a predetermined thickness to the surface of the silicone sponge on which the adhesive is coated and adequately shaping the bag.

8. A method for manufacturing a lightweight silicone implant comprising the steps of:
(a) making vacant silicone capsules having a desired shape by combining a plurality of small silicone capsules and small silicone pieces at high temperature;
(b) coating a spray liquid adhesive on a surface of the vacant silicone capsules; and
(c) adhering a silicone bag or a cohesive gel bag having a predetermined thickness to the surface of the vacant silicone capsules on which the adhesive is coated and adequately shaping the bag.

9. The lightweight silicone implant according to claim 2, wherein the cohesive gel bag includes a silicone bag and a cohesive gel contained in the silicone bag.

10. The lightweight silicone implant according to claim 2, wherein the silicone bag or the cohesive gel bag has a thickness of 0.3 mm to 5 mm.

11. The lightweight silicone implant according to claim 2, wherein the silicone bag or the cohesive gel bag is coupled to the foamed silicone sponge or the silicone capsules by use of a spray liquid adhesive.
